(19) United States
(12) Patent Application Publication
(10) Pub. No.: US 2006/0047291 A1
(43) Pub. Date: Mar. 2, 2006

(54) Non-foreign occlusion of an airway and lung collapse

(75) Inventor: Robert L. Barry, Kirkland, WA (US)

Correspondence Address: UPTAKE MEDICAL CORPORATION, 2028 5TH AVENUE, SUITE 204, SEATTLE, WA, 98121, US

(73) Assignee: Uptake Medical Corporation, Seattle, WA (US)

(21) Appl. No.: 11/208,396

(22) Filed: Aug. 20, 2005

Related U.S. Application Data
(60) Provisional application No. 60/602,931, filed on Aug. 20, 2004.

(51) Int. Cl. A61B 17/22

(52) U.S. Cl. 606/159

(57) ABSTRACT

Methods, devices and systems for the achieving lung volume reduction by non-foreign occlusion of a patient's airways and inducing neointima hyperplasia to occlude the airways.

### CROSS REFERENCE

This application claims the benefit of U.S. Provisional Application No. 60/602,931, filed Aug. 20, 2004, which is incorporated herein by reference in its entirety.

### FIELD OF THE INVENTION

The present invention is related to the medical devices, systems, methods and kits for lung volume reduction (LVR) of a predetermined region of a patient's lung, and more specifically, for the non-foreign occlusion of one or more airways to achieve LVR.

### BACKGROUND OF THE INVENTION

Emphysema is a debilitating disease. A subtype of chronic obstructive pulmonary disease (COPD), emphysema is characterized by the destruction of the lung parenchyma, which leads to the primary pathology of emphysema, namely the dilatation and destruction of respiratory bronchioles, subsequent gas exchange abnormalities and eventual pulmonary hypertension and right heart failure as the disease progresses.

In addition to medical management, lung volume reduction surgery (LVRS) is used to remove damaged lung tissue and is a treatment for patients with emphysema as well as other lung disorders. In this surgical procedure, about 20-30% of a patient's total lung volume is excised. While several clinical studies have shown the effectiveness of LVRS, this procedure is fairly expensive and the risks of early post-operative mortality are high in patients who are compromised by lung disease.

Recently, non-surgical, bronchoscopic approaches for achieving lung volume reduction have been proposed. For example, U.S. Pat. Nos. 6,904,909; 6,901,927 and 6,860,847 describe the implantation of endobronchial valves. In these approaches, a mechanical device or other device is implanted into a patient's airway prevents inspired air from inflating a diseased, pre-selected portion of a lung.

Over time, the treated lung region deflates or eventually become atelectatic. Other approaches to bronchoscopic lung volume reduction that have been recently described include the use of endobronchial sealants, sealants and plugs and fenestrations and bypass. Unfortunately, most of these procedures can be technically difficult and leave behind foreign objects that could dislodge or migrate, cause erosion and/or tearing of a patient's airway. Moreover, to occlude a particular targeted lung region, one or more valves or plugs may have to be used to treat a targeted region, increasing the cost of the treatment procedure to the patient.

What is needed therefore is a less invasive method for closing or occluding an airway, which does not have the associated risk of an open chest procedure, is technically easy to perform, and which causes efficient occlusion of an airway and resulting atelectasis in a treated lung region without leaving any foreign material behind. The present invention is directed at meeting these as well as other needs.

### SUMMARY OF THE INVENTION

The present invention is related to the non-foreign occlusion of an airway, to achieve lung volume reduction, by induction of a controlled injury to airways and adjacent lung tissues. Controlled trauma or injury can be used to induce a healing process, which results in neointima hyperplasia and proliferation of smooth muscle cells, as well as chronic inflammation and wall thickening via remodeling; hypertrophy and thickening of the airway walls; and eventual tissue fibrosis (in addition to a myriad of other processes).

In the present invention, various methods, devices and systems are provided to induce neointima hyperplasia in order to partially or totally occlude one or more airways is provided. As with the mechanical valves and other occlusive procedures, occlusion of one or more airways results in its eventual atelectesis and lung volume reduction of a pre-selected lung region.

Methods, devices and systems to induce trauma to occlude an airway and injury to surrounding lung tissues (i.e. lung parenchyma) are also provided as an approach to achieve atalectesis of an airway as well as atalectesis and LVR of a pre-selected lung region (ie. a lobe or subset of a lobe). In a preferred embodiment, concomitant trauma to an airway and surrounding paranchymal tissues is acute and sufficient to create acute shrinkage and occlusion of an airway (due to collagen shrinkage), followed by atelecteis and fibrosis of the injured paranchymal tissues and pre-selected lung region. In yet another aspect of the invention, the injury can be limited to lung tissues or alternatively be targeted to include adjacent structures including blood vessel and capillaries so necrosis of tissues in a target lung region includes cessation of blood supply to the pre-selected lung region.

As is further described herein, methods, devices and systems for inducing neointima hyperplasia mechanically, chemically (i.e. talc, chemical irritant), enzymatically or via the deposition or removal of energy (RF, laser, microwave, cryo, RF, high intensity ultrasound, hot air or liquid, or most preferably via vapor deposition) are also provided.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic diagram of patient's lung;

FIG. 2 is a cross-sectional view of an airway of FIG. 1, taken along line 2-2;

FIG. 3 is a schematic diagram of a lung being treated with a treatment catheter of the present invention advanced to a treatment location in a lobar, segmental or subsegmental airway in accordance with the present invention;

FIG. 4 is a schematic diagram illustrating one embodiment of the invention wherein an airway is mechanically abraded thereby partially or completely occluding the airway;

FIG. 5 is a schematic diagram illustrating one embodiment of the invention wherein an airway is partially or completely occluded via thermal injury of the inner airway lumen;

### DETAILED DESCRIPTION OF THE DRAWINGS

The present invention provides novel, non-invasive devices (catheters), methods and systems for injuring airways and lung tissue to achieve lung volume reduction.

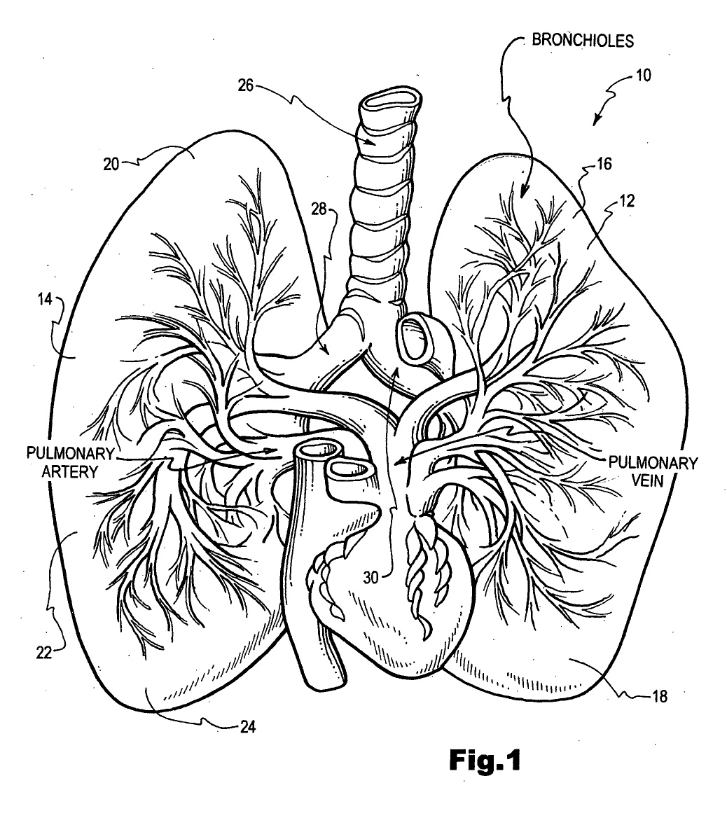

FIG. 1 illustrates human lungs 10 having a left 12 and right 14 lung. The left lung 12 is divided into two lobes, the superior 16 and inferior 18 lobes. The right lung 14 is divided into the superior 20, middle 22, and inferior 24 lobes. The trachea 26 extends down from the larynx and conveys air to and from the lungs 10. The trachea 26 divides into the right and left main bronchi (28, 30) as it reaches the right and left lung respectively. Within the right and left lungs, the main bronchi taper and turn into lobar, segmental and subsegmental bronchi or airway passageways, which finally terminate at the terminal bronchioles and the alveoli.

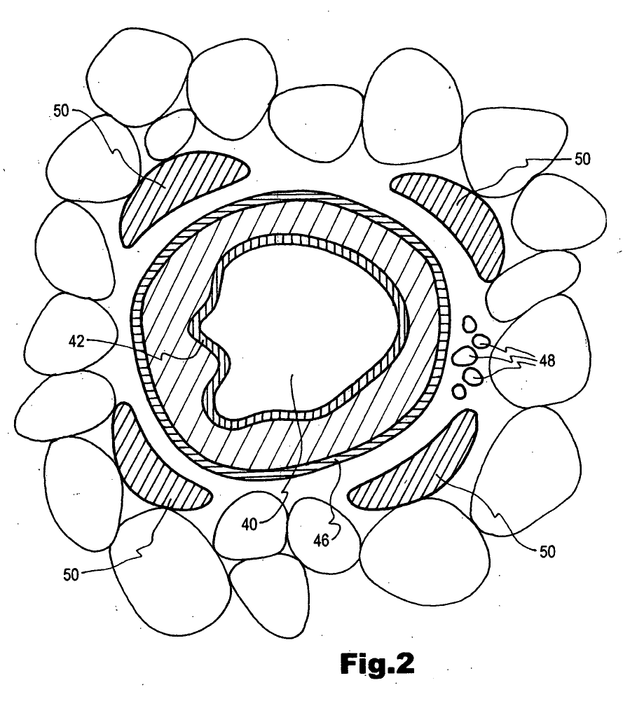

FIG. 2 illustrates a cross-sectional view of an airway 40 through which gases are inhaled and exhaled. The airway 40 includes an inner surface of epithelium 42 surrounded by stoma 44, smooth muscle tissues 46, mucous glands 48 and cartilage 50. Generally, blood vessels run along side the major lung airways 40. In the alveolar level, where gas exchange occurs, there are capillary beds.

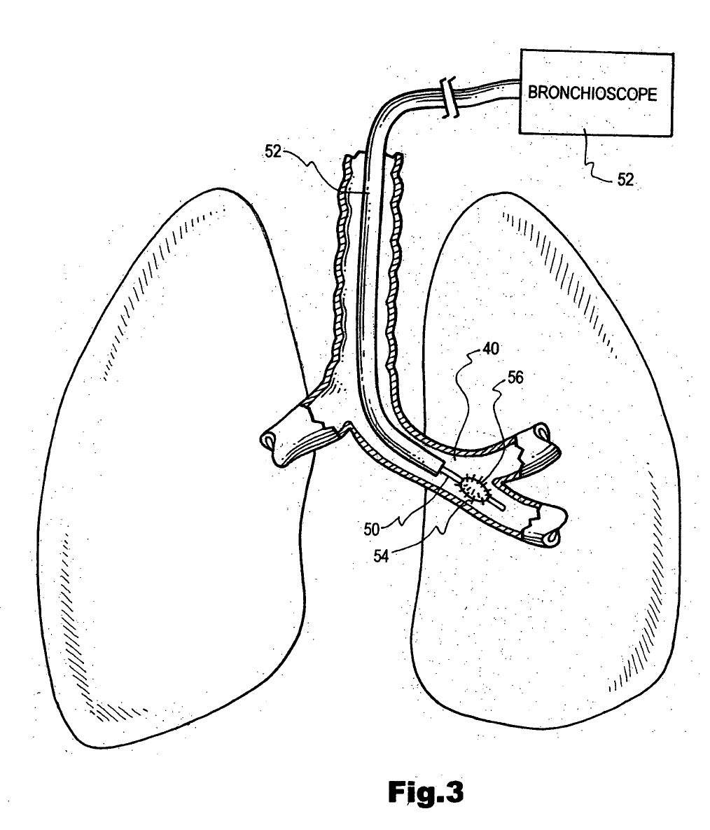

FIG. 3 is a schematic diagram of a lung 10 being treated with a treatment catheter 50, of the present invention, advanced to a treatment location in a segmental or subsegmental airway in accordance with one embodiment of the invention. In a preferred embodiment, the catheter 50 is adapted to be used in conjunction with a pulmonary bronchoscope 52 and advanced through the working channel (not shown) of the scope 52. It should be generally understood that the treatment catheter 50 of the present invention can be advanced through a scope into a lobar, segmental or subsegmental airway.

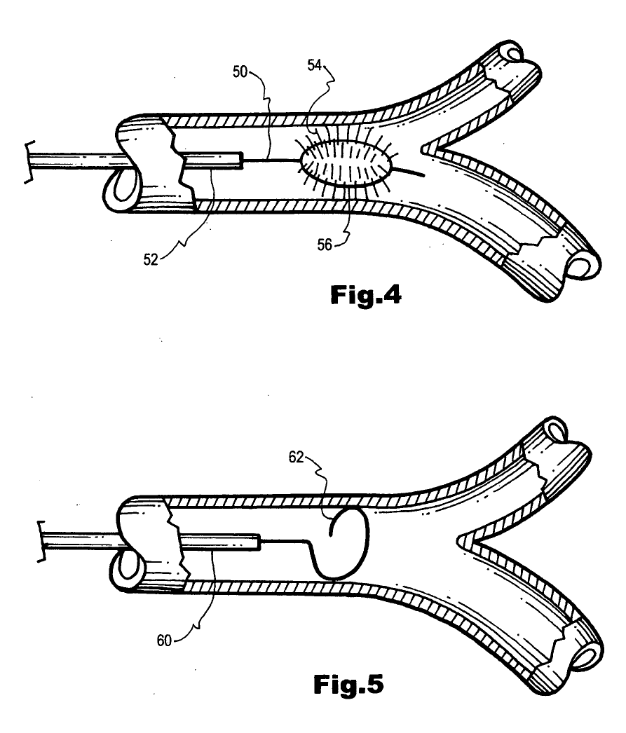

As illustrated in FIG. 4, in one embodiment of the invention, treatment catheter 50 comprises a plurality of abrasive members 54. These abrasive members 54 (such as wires (plastic or metallic), glues or the like, in one embodiment can be attached over an inflatable balloon 56. The balloon 56 is inflated while in the airway 40 and the abrasive members 54 forced into inner lumen tissue of the airway. The user can then remove endothelial cells and create trauma and injury to the airway by rotating and or otherwise moving the abrasive members 54 disposed therein. The balloon 56 is then deflated and then the treatment catheter is retracted into the scope 52.

Once the endothelium has been removed and the area inside the airway traumatized, the body's healing mechanism begins. The body's healing mechanism then, in a preferred embodiment, replaces the traumatized tissue with fibrotic scar tissue across the injured airway, preventing ventilation into the airways distal to the scar tissue formation. Over time, the airway and the pre-selected lung region ventilated by the airway becomes atelectatic and collapsed due to the lack of ventilation of the lung region, thus achieving lung volume reduction. In addition, by occluding and preventing ventilation of the target lung region containing diseased tissues, inspired air is redistributed to better function regions of a patient's lungs.

In yet another embodiment, acute closure of an airway is induced by sufficiently irritating and injuring of the airways with treatment catheter to induce acute collapse and eventual scarring down and occlusion of the treated airway.

In yet another embodiment of the invention, in addition to mechanical abrasion, other methods of injuring airways to induce injury induce occlusion of airways are within the scope of the present invention, including chemical or enzymatic insult to the airways. In one embodiment, talc can be introduced to cause chemical irritation and injury.

FIG. 5 illustrates yet another embodiment of the present invention wherein energy deposition can induce a thermal injury and airway occlusion. As shown in one embodiment, treatment device 60 can include one or more RF electrodes 62 disposed at the distal end of the treatment catheter 60. For example, RF electrode 62 may be in the form of an electrically conductive wire 62, coil or other member disposed at the distal end of the treatment catheter 60. Alternatively, the one or more electrode 62 can be disposed on an outer surface of an inflation member or a balloon (not shown).

In one embodiment, the one or more RF electrode 62 can be use to deliver sufficient thermal energy to heat injury the airways (i.e. the inner lumen of the airway) or alternatively sufficient energy delivery to induce thermal injury of the airways and surrounding vascular structures and a region of parenchymal lung tissue. Deposition of sufficient thermal energy (typically enough to raise native tissue temperatures above 45 degrees C. and more preferably above 65 degrees C.) will injure and traumatize the adjacent cells and tissue and initiate a healing response and resulting neointima hyperplasia in the airways as well as necrose and scar down the pre-selected lung region. As ventilation and arterial perfusion of the cells in the target lung region is prevent by thermally induced airway occlusion and tissue necrosis, these mechanisms will be sufficient to permanently prevent re-inflation of a thermally treated lung region.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed:

1. A treatment catheter for abrading an airway comprising:
an elongated catheter having proximal and distal ends and an inflatable balloon located on the distal end of the catheter; and
said inflatable balloon adapted to be disposed within the airway and having a plurality of members for injuring an inner lumen of the airway sufficient to induce neointima hyperplasia and occlusion of said airway.

2. The catheter of claim 1, wherein the members are adapted to mechanically abrade the inner lumen of the airway.

3. The catheter of claim 1 wherein the members are one or more energy delivery members adapted to thermally injure the inner lumen of the airway.

4. The catheter of claim 3 wherein the members are one or more RF electrodes.

5. The catheter of claim 3 wherein the member are adapted to deliver energy to tissue surrounding the airway.

6. The catheter of claim 5 wherein the member are adapted to sufficient heat the surround tissue to a temperature sufficient to shrink collagen.

7. A method of occluding a patient's airways to achieve lung volume reduction, said method comprising:
advancing a bronchoscope into an airway and positioning treatment catheter into a segmental or subsegmental airway of a patient;
traumatizing inner lumen of the airway in order to induce neointima hyperplasia in the airway and partially or completely occlude the airway.
